(19) United States
(12) Patent Application Publication
(10) Pub. No.: US 2024/0393285 A1
(43) Pub. Date: Nov. 28, 2024

(54) COMPOSITIONS FOR USE IN THE TREATMENT OF MUSCULOSKELETAL CONDITIONS AND METHODS FOR PRODUCING THE SAME LEVERAGING THE SYNERGISTIC ACTIVITY OF TWO DIFFERENT TYPES OF MESENCHYMAL STROMAL/STEM CELLS

(71) Applicant: REGENERA SCIENCES IP, LDA., COIMBRA (PT)

(72) Inventors: Ana SANTOS, COIMBRA (PT); Sílvia PEDROSA, COIMBRA (PT); Mariana BRANQUINHO, COIMBRA (PT); Rui ALVITES, COIMBRA (PT); Ana MAURICIO, COIMBRA (PT); Sergio CAMOES, COIMBRA (PT); Joana RODRIGUES, COIMBRA (PT); Joana MIRANDA, COIMBRA (PT); Jorge SANTOS, COIMBRA (PT); Ines REIS, COIMBRA (PT)

(21) Appl. No.: 18/793,848

(22) Filed: Aug. 4, 2024

Related U.S. Application Data
(63) Continuation of application No. 16/997,106, filed on Aug. 19, 2020.

(30) Foreign Application Priority Data
Mar. 12, 2018 (PT) 110617

(51) Int. Cl. G01N 27/30; G01N 27/12; G01N 33/00

(52) U.S. Cl. CPC G01N 27/30; G01N 27/125; G01N 33/0009

(57) ABSTRACT

The present invention relates to compositions comprising mesenchymal stem cells (MSC) useful for producing pharmaceutical formulation to treat musculoskeletal conditions, including joint degeneration, tendon and ligament laxity or rupture, and muscle conditions.

The present invention relates to the field of regenerative medicine, namely formulating improved compositions for the treatment of musculoskeletal conditions, including joint degeneration, tendon and ligament laxity or rupture, and muscle conditions, having a positive impact on pharmaceutical formulations containing mesenchymal stem cells (MSCs), providing the enhancement of MSCs viability upon cryopreservation, MSCs stability during transportation and manipulation, and MSCs therapeutic efficacy of the final formulations.

### FIELD OF THE INVENTION

The present invention relates to compositions comprising mesenchymal stem cells (MSC) useful for producing pharmaceutical formulation to treat musculoskeletal conditions, including joint degeneration, tendon and ligament laxity or rupture, and muscle conditions.

The present invention relates to the field of regenerative medicine, namely formulating improved compositions for the treatment of musculoskeletal conditions, including joint degeneration, tendon and ligament laxity or rupture, and muscle conditions. More specifically, the present invention have positive impact on pharmaceutical formulations containing mesenchymal stem cells (MSCs), providing the enhancement of MSCs viability upon cryopreservation, MSCs stability during transportation and manipulation, and MSCs therapeutic efficacy of the final formulations. The invention is to be applied in the production of either human or veterinary MSCs-based pharmaceutical formulations for the treatment of the formerly mentioned pathologies, whether resulting from age-related degeneration, continued use-related degeneration, effort-related degeneration, illness-related degeneration, congenital condition-related degeneration, osteoarthritic symptoms in general and accident-related tear out causing acute inflammation.

### BACKGROUND OF THE INVENTION

Degenerative joint disease (DJD) is a condition that results in the degeneration of the cartilage tissues that line and lubricate the joints in the body. Once the cartilage is thinned or torn, the continued friction will lead to osteoarthritis (OA). The result is chronic inflammation, resulting in swelling and pain, with subsequent loss of joint function. Due to a unique tissue composition and architecture, basically consisting on a single chondrogenic phenotype, the articular cartilage mostly receives nutrition through diffusion from the synovial fluid, resulting in a limited intrinsic capacity for healing. This affects both articular cartilage and adjacent bone.

The pain and stiffness caused by OA are first associated with decreased joint range of movement, thereafter the disease may rapidly evolve to irreversible loss of function. In more severe cases, spurs grow from damaged articular bones, causing further pain and stiffness, and lesions to adjacent tissues like cartilage, synovium or muscle.

The origin of OA is often attributed to cartilage wear out mainly provoked by continued friction with loss or reduced amounts of synovium fluid. Excessive (associated with obesity) or uneven (from congenital or acquired articular axis deviations) load bearing, as well as muscular and ligament abnormalities also play a role in cartilage overload, contributing to the development and progression of the degenerative affection.

Research has shown that laxity in the bone/ligament insertion leads to subchondral bone changes. Such changes provoke cartilage defects that in turn lead to OA. This is a relevant situation in an increasingly aging society, resulting in early loss of quality of life. This is also particularly serious in younger age groups, resulting from accidents, as well as in high performance athletes, leading to precocious retirement form the professional as sports activities.

Just like chronic degeneration leading to OA, acute cases of joint tissue tear out, such as lesions inflicted by overstress or accident, have no curative treatments. The standard of care is still mainly restricted to symptom management using hyaluronic acid infiltrations, nonsteroidal anti-inflammatory drugs (NSAIDs), cortisone administration and ultimately arthroscopic surgery. NSAID overdose, cortisone and other steroid-based treatments have undesirable adverse effects.

Ultrasound has been found to be effective in accelerating the healing process, however it does not increase the intrinsic regenerative capacity of the body (WO2015/017772 A1, PCT/US2014/049395).

Other treatments include ultrasound and the use of platelet rich plasma (PRP), which is a concentrate of platelet-rich plasma proteins derived from whole blood, centrifuged to remove red blood cells. It has a greater concentration of growth factors than whole blood, and it has been used to induce the intrinsic regenerative capacity of joint soft tissues as adjuvant to resident precursor/stem cells.

Globally, the results are inconsistent and non-conclusive. PRP effects are highly dependent on the fact there is still endogenous regenerative capacity of the lesioned tissues, which in turn is related to the number and type of progenitor/stem cells that can still be recruited to the regenerative process.

Cell therapy has emerged as real regenerative alternative to the current standard of care for musculoskeletal conditions. Cell therapy can offer potential to fully recover healthy tissue properties and functionalities.

Apart from the strategy of autologous transplantation of chondrocytes, which by itself has not fully achieved newly-formed cartilage with the same mechanical properties as natural tissue, the administration of stem cells has been extensively used.

Amongst the stem cells that are being proposed as active substance for advanced medicinal products, MSCs have emerged as the most promising regenerative agents given their engraftment and differentiation capacity thus replacing injured or diseased cells and tissues (Bárcia et al., 2015).

However, several reports demonstrate that the positive action of MSCs on the tissue remodelling process is not only via differentiation and cell replacement, but also through the paracrine secretion of trophic factors, which in turn is mediated by environmental signals. Secreted paracrine factors, like cytokines and other growth factors, can promote cell-to-cell chemotactic interactions that modulate inflammation, immune reactions and the fate of surrounding cells towards healing pathways, thus augmenting the regenerative response of the organism (Santos et al., 2015).

MSCs are long-lived cells that coexist undifferentiated in most tissues throughout the human body and contribute largely to the intrinsic regenerative capacity of the organism, maintaining homeostasis and repairing damage upon abrasion or wounding. MSCs have been identified in synovial tissues, including those of the diarthrodial joint, and cartilage with potential for cartilage regeneration in vivo. This suggests that synovial tissues have a resident MSCs population that increases in response to tissue damage, such as OA-related. MSCs are also found in higher number in synovial fluid from knees after meniscal injury than in normal ones).

A comparative study between different types of MSCs has shown that while adipose tissue-derived MSCs (AT-MSCs) were superior in terms of inducing adipogenesis, and bone marrow-derived MSCs (BM-MSCs) were superior for promoting osteogenesis, MSCs derived from synovium tissues (SM-MSCs) had the greatest chondrogenic potential, representing a possible therapeutic agent for cartilage repair.

SM-MSCs, more committed to a chondrogenic lineage than other types of MSCs, naturally contribute to maintenance of healthy joints by acting as reservoirs of repair cells or as immunomodulatory sentinels to reduce joint inflammation. In fact, the onset of degenerative changes in the joint is associated with aberrant activity or depletion of these cell reservoirs, leading to loss of chondrogenic potential and preponderance of a fibrogenic phenotype. Functional studies showed that SM-MSCs were distinct from BM-MSCs.

SM-MSCs form a pool of highly clonogenic cells with chondrogenic potential, whereas BM-MSCs are very heterogeneous. Another study revealed that SM-MSCs originated neither from bone marrow nor from circulating MSCs, but from the synovial membrane or cartilage. The local replenishing of ex vivo cultured MSCs derived from synovial tissues has produced promising outcomes in preclinical models of joint disease and it has been subject of patenting as a method to treat osteoarthritis.

Further to the presence of MSCs committed to the chondrogenic lineage, evidence has been produced for the presence of endothelial precursor cells, which can induce new vessel formation in the synovial tissue of patients with rheumatoid arthritis (RA) and OA. Namely, a population of CD34+, CD31− cells was detected close to STRO-1+ and CD133+ cells, forming cell clusters in the sub-lining area of the synovial membrane. The authors conclude that the presence of endothelial precursor cells in the synovial tissue of RA and OA patients provides evidence for vasculogenesis induced by precursor cells that arise in situ or from circulating progenitors.

In turn, of all MSCs being applied in preclinical and clinical studies worldwide, the use of the umbilical cord tissue-derived MSCs (UC-MSCs), such as the UCX® cellular product (PCT/IB2008/054067; WO 2009044379) has clearly demonstrated the difference between the therapeutic potential of MSCs obtained from different sources. UCX® cells, for example, were shown to repress T-cell activation and promote the expansion of Tregs better than bone marrow-derived mesenchymal stem cells (BM-MSCs). Consequently, xenogeneic UCX® administration in an acute carrageenan-induced (ACI) arthritis murine model showed that UCX® cells (which are human) can reduce rat paw oedema in vivo more efficiently than BM-MSCs. Furthermore, in a chronic adjuvant induced arthritis model, rats treated with intra-articular and intra-peritoneal infusions of UCX® cells showed faster remission of local and systemic arthritic manifestations (Santos et al., 2013).

In another study, UCX® cells were shown not to need prior activation or priming to exert their immunomodulatory effects in contrast with BM-MSCs. This was further corroborated in vivo in the CIA model of acute inflammation. In this same study, the potency differences observed between UCX® and BM-MSCs were elucidated at the level of gene expression. Several gene expression profile differences were found between UCX® and BM-MSCs in the same growth conditions, namely decreased expression of HLA-DRA, HO-1, IGFBP1, 4 and 6, ILR1, IL6R and PTGES and increased expression of CD200, CD273, CD274, ILiB, IL-8, LIF and TGFB2. The latter were also confirmed at the protein expression level (Bárcia et al., 2015).

More recently, the paracrine activity of UC-MSCs like UCX® was further emphasized. UCX® cells were shown to preserve cardiac function upon intra-myocardial transplantation in an acute myocardial infarction (AMI) murine model. The cardio-protective effects of UCX® were attributed to paracrine mechanisms that promoted angiogenesis, limited the extent of the apoptosis in the heart, augmented proliferation and activated a pool of resident cardio progenitor cells (Nascimento et al., 2014). In turn, a UCX® three-dimensional culture model, which better mimics the in vivo physiological conditions, was developed and characterized with respect to spheroid formation, cell phenotype and cell viability. The secretion by UCX® spheroids of extracellular matrix proteins and trophic factors involved in the wound-healing process was analysed. The skin regenerative potential of UCX® three-dimensional culture-derived conditioned medium (CM3D) was also assessed in vitro and in vivo against UCX® two-dimensional culture-derived conditioned medium (CM2D) using scratch and tubulogenesis assays and a rat wound splinting model, respectively. The UCX® spheroids kept in three-dimensional cultures remained viable and multipotent and secreted considerable amounts of vascular endothelial growth factor A (VEGF-A), which was undetected in CM2D, and higher amounts of matrix metalloproteinase-2 (MPP2), matrix metalloproteinase-9 (MPP9), hepatocyte growth factor (HGF), transforming growth factor B1 (TGF− B1), granulocyte-colony stimulating factor (G-CSF), fibroblast growth factor 2 (FGF2) and interleukin-6 (IL-6), when compared to CM2D.

Furthermore, CM3D significantly enhanced elastin production and migration of keratinocytes and fibroblasts in vitro. In turn, tubulogenesis assays revealed increased capillary maturation in the presence of CM3D, as seen by a significant increase in capillary thickness and length when compared to CM2D, and increased branching points and capillary numbers when compared to basal medium.

Finally, CM3D-treated wounds presented signs of faster and better resolution when compared to untreated and CM2D-treated wounds in vivo. Although CM2D proved to be beneficial, CM3D-treated wounds revealed a completely regenerated tissue by day 14 after excisions, with a more mature vascular system already showing glands and hair follicles (Santos et al. 2015).

Finally, the paracrine activity towards tissue regeneration of UCX® cells was confirmed in vitro using chemotaxis assays and an in vivo transplantation model for chemoattraction. The results confirmed that UCX® are chemotactic to CD34−/CD45− BM-MSCs via a one-way, cell-specific, mobilization mechanism mediated by G-CSF; revealing the potential of UCX® to extend the regenerative capacity of the organism by complementing the role of endogenous BM-MSCs (Miranda et al., 2015).

Many MSCs-based pharmaceutical compositions for the treatment of joint tissues can be found in the scientific literature, with higher emphasis on cartilage. In a recent systematic review by Goldberg and colleagues (Goldberg et al., 2017), the authors found that BM-MSCs were the most often used type of MSC for cartilage repair in animal pre-clinical studies (84 studies found in the literature corresponding to 75%), followed by AT-MSCs (13 studies corresponding to 11%). Out of 109 studies found, only 6 used synovia-derived MSCs (5%) and no studies were found using UC-MSCs (Nakamura et al., 2012; Lee et al., 2012 and 2013; Pei et al., 2013).

According to the same systematic review, MSCs were transplanted into the defects both as cell therapy (injection directly into the joint) (17 studies, 15%) or by tissue engineering (cell-scaffold combinations) (94 studies, 85%). Fifteen studies used a mixture of MSCs combined with solutions prepared from hyaluronic acid, phosphate buffer, collagen acid or sodium alginate. Of interest for this invention are the two studies where either SM-MSCs or growth factor medium additives were used, (Iwai et al. 2011) or a combination of SM-MSCs with platelet rich plasma (PRP) (Lee et al. 2013).

In the first study, Iwai and colleagues aimed at evaluating the viability of a new chondral disc model to evaluate the effects of medium additives such as serum, transforming growth factor-β3 (TGF-β3), fibroblast growth factor-2, and the transfection of TGF-β3 gene to MSCs (Iwai et al. 2011). In the second study, Lee and colleagues embedded SM-MSCs in a platelet-rich-plasma (PRP) gel as a strategy to resurface the cartilage defect and restore the subchondral bone in a rabbit model.

In both studies, i) the only MSCs activity involved was the one derived from SM-MSCs, ii) platelets or growth factors were not used during MSC preparation, and iii) the nature of adjuvant growth factors was not clearly defined and their cumulative effects, if any, were not quantified.

In turn, at the human clinical trial level, 21 studies so far (68%) used BM-MSCs from the anterior or posterior superior iliac spine to regenerate joint cartilage; 5 studies (18%) used AT− MSCs; 2 studies (7%) used peripheral blood progenitor cells collected by apheresis. Finally, 2 studies (7%) used SM-MSCs (Akgun et al., 2015; Sekiya et al., 2015; Goldberg et al., 2017). In these two studies, SM-MSCs were used alone (either matrix-pre-induced or not) without adjuvants, showing good effectiveness and potential to accelerate recovery of full-thickness chondral lesions of the knee (Akgun et al., 2015; Sekiya et al., 2015).

Other studies have suggested that MSCs treatment can regenerate tendons and ligaments through the neo-formation of fibrocartilagenous tissue rather than scar tissue. MSCs treatment for lower limb tendons have resulted in improvements in some studies using BM-MSCs but also SM-MSCs (Ju et al. 2008, Nourissat et al. 2010). Achilles tendons injected with SM-MSCs demonstrated improved collagen fiber appearance as early as 1 week after treatment (Ju et al. 2008).

Recently, Chamberlain et al. preconditioned allogeneic BM-MSCs to be even more anti-inflammatory (using TNF-α) and used them in rat Achilles tendon and MCL healing models. When compared with normally processed (unconditioned) MSCs, pre-conditioned MSCs significantly reduced inflammation by increasing the M2 macrophages and decreasing the M1 macrophages. Most importantly, treatment with pre-conditioned MSCs improved tissue strength when compared to unconditioned MSCs (Chamberlain et al., 2017).

In turn, application of either SM-MSC or UC-MSC alone, or of their derivate products (SM-MSC-CM or UC-MSC-CM), has been reported as beneficial in experimental settings of several muscle lesions, improving both structural and functional recovery (De Bari, Dell'Accio et al. 2003, Conconi, Burra et al. 2006, de la Garza-Rodea,).

In turn, patent documents disclosing protecting methods of isolation, purification and industrial scale expansion of human, equine and canine adipose tissue-derived MSCs. These also relate to methods for treating tendon injury, ligament injury, osteoarthritis, exercise-induced pulmonary haemorrhage, idiopathic pulmonary fibrosis, multiple sclerosis, Duchenne muscular dystrophy, rheumatoid arthritis, spinal cord injury, atopic dermatitis, dilated cardiomyopathy, type-1 and type-2 diabetes, renal failure, hepatic disease, critical limb ischemia, cerebral stroke and non-healing wounds (WO/2014/203267, WO/2014/203268, WO/2014/203269).

Another relevant patent documents refer to the use of adherent cells derived from a tissue selected from the group consisting of placenta and adipose tissues, or the use of the medium conditioned by such adherent cells, for use in treating stem cell deficiency, heart disease, Parkinson's disease, cancer, Alzheimer's disease, stroke, burns, loss of tissue, loss of blood, anaemia, autoimmune disorders, diabetes, arthritis, graft vs. host disease (GvHD), neurodegenerative disorders, autoimmune encephalomyelitis (EAE), systemic lupus erythematosus (SLE), rheumatoid arthritis, systemic sclerosis, Sjorgen's syndrome, multiple sclerosis (MS), myasthenia gravis (MG), Guillain-Barre syndrome (GBS), Hashimoto's thyroiditis (HT), Graves's disease, insulin dependent diabetes mellitus (IDDM) and inflammatory Bowel disease (EP2626417).

Another patent document refers to the use of MSCs for articular cartilage repair when combined with a controlled-resorption biodegradable matrix in their use for repair of cartilage damaged as part of the degenerative effects of osteoarthritis. Although it is not specified the nature or type of stem cells to be used, the examples show a single type of MSC, namely autologous osteochondral precursors (EP 2110431).

Yet, EP1276486 refers to the use of MSCs and an acceptable pharmaceutical carrier for the preparation of an injectable pharmaceutical composition for regeneration or reparation of meniscal tissue in a joint, wherein said injectable pharmaceutical composition comprises an effective amount of MSCs for regeneration or reparation of meniscal tissue in a joint and is to be injected into the joint space; and wherein said MSCs differentiate into or stimulate production of meniscal tissue. The same patent still claims the nature of the said carrier as being hyaluronan or a chemically modified hyaluronan, as well as the fact that the said MSCs are allogeneic to the recipient.

Finally, US 0241144 refers to the use of MSCs or synovial fluid, or the mixture thereof, for the treatment and prevention of osteoarthritic conditions. This document does not disclose the existence of MSCs in the said synovial fluid which, as per the authors, can be synthetically prepared with no mention to a biological composition.

In summary, although much is known about the differences between different types of MSCs, their use in cell therapy still lacks precise formulation definitions—one of the major regulatory hurdles that have been hindering MSC translation from bench to bedside.

Besides, and although much is known about the differences between different types of MSCs, there are still a lack of proposals considering therapeutic formulations where the complementary activity of two (or more) different types of MSCs is considered to the level of understanding the relative potential of the cellular and molecular actors involved, and where those actors could make a difference along the production process with respect to a specific application.

The present invention provides methods to overcome relevant problems of MSC formulation preparation and manipulation by providing means to improve cell viability upon cryopreservation, increase the therapeutic activity based on cumulative synergistic effects between two different types of MSCs and significantly improve MSC stability at 4° C. and room temperature.

### SUMMARY OF THE INVENTION

The present invention relates to compositions comprising mesenchymal stem cells (MSCs) useful for producing pharmaceutical formulation to treat musculoskeletal conditions, including joint degeneration, tendon and ligament laxity or rupture, and muscle conditions.

Therefore, in one aspect, the present invention describes a composition comprising cells and/or cell-based active substances obtained from more than one body tissue producing mesenchymal stem/stromal cells according to claim 1.

In another aspect, the present invention describe a composition comprising cells and/or cell-based active substances obtained from more than one body tissue producing mesenchymal stem/stromal cells to be used as medicament for humans or animals according to claim 7.

In another aspect, the present invention describes a method of treatment of a musculoskeletal condition, an osteoarthritis condition or any other condition etiologic to osteoarthritis, and/or a tendinopathy condition or any other condition etiologic to tendinopathy in humans or animals by using a composition comprising cells and/or cell-based active substances obtained from more than one body tissue producing mesenchymal stem/stromal cells according to claim 11.

In another aspect, the present invention provides a process for producing a composition comprising cells and/or cell-based active substances obtained from more than one body tissue producing mesenchymal stem/stromal cells according to claim 12.

A grand limitation on the development and dissemination of cellular therapies is the limited numbers of cells that can be isolated, when compared to the large numbers of cells required for therapies. To achieve this number of viable cells, they must be expanded, a process restrained to a limited number of population doublings due to cellular senescence, loss of safety assurance and therapeutic power. Additionally, the cryopreservation is an essential step in most cellular manipulation processes that is often associated with decreased cell viability and cell numbers upon thawing. When compared to the currently freeze/thawing protocols, the invention can increase the number of viable cells recovered (e.g. from a limited master cell bank (MCB) or single donor sample) upon thawing, thus contributing significantly to increase the number of viable stem cell banks, and therefore of expanded cell dose numbers.

Currently, existing options focus on the application of one MSCs type or its derived products, providing only the beneficial effects attributed to each method.

The present invention proposes means with potential to improve the efficacy of stem cell-based musculoskeletal tissue regeneration through the combination of two active compounds in one single administration, and also the efficiency of such treatment approaches due to increased number of viable cells. Prospective joint application of the MSC and/or derivate products increases motility capacity of surrounding synoviocytes (an important mechanism involved in joint tissue healing/regeneration), induces glycosaminoglycan (GAG) production by surrounding chondrocytes (essential for new cartilage formation) and has the capacity to reduce lesion size. Regarding tendon and ligament lesions, MSCs and/or derivate products increases fibre density, improves fibre alignment and tendon integrity in vivo when compared to the independent application of the components—the best current procedures, using synovial mesenchymal stem cells alone.

Finally, the invention has the capacity to solve one of the major logistical problems around cell therapy, which is transportation of readily applicable formulations with increased cell viability. In fact, many cell therapy protocols, namely in the veterinarian area, do not comply with cryo-transportation requirements, which is expensive and sometimes logistically impossible. The invention has the important advantage to considerably increase cell viability within the formulation through time, at 4° C. and room temperature (RT −22° C.), when compared to currently formulation protocols, such as autologous serum, making it possible to prolong the survival of a cell dosage e.g. for 48 h at room temperature, without significant loss in cell viability.

This achievement widens the list of ultimate beneficiaries for the administration of the therapy by practitioners in smaller clinics (or even in ambulatory practice in the case of veterinary medicine), that are currently bound to large medical facilities.

### DESCRIPTION OF THE FIGURES

FIG. 1: Detection and Quantification in pg/mL of cytokines, chemokines and growth factors, through Multiplexing LASER Bead Analysis (Eve Technologies, Calgary, Alberta, Canada) in hUCBP presented as Mean±SE.

FIG. 2: Relative quantitative distribution of the metabolites in hUCBP presented as Mean±SE.

FIG. 3

FIG. 3.1 SM-MSCs viability upon thawing using either AS or UCBP, after cryopreservation in 90% FBS or UCBP: 10% DMSO. Significant differences are indicated according to P values with one, two, three or four of the symbols (*) corresponding to 0.01<p≤0.05, 0.001<p≤0.01, 0.0001<p_0.001 e p<0.0001, respectively.

FIG. 3.2 Experimental design for the Freeze-Thaw viability assessment of SM-MSCs.

FIG. 4: Percentage of reduction in the scratched area of SM-MSCs under DMEM-F12 10% FBS, SM-MSC-CM and/or UC-MSC-CM stimuli, after 19, 26 and 43 hours. Statistical differences were considered at P<0.05, and significance of the results is indicated according to P values with one, two, three or four of the symbols (*) corresponding to 0.01≤P<0.05; 0.001≤P<0.01; 0.0001≤P<0.001 and P<0.0001, respectively.

FIG. 5: Microphotographs and % of area reduction of the scratched area of SM-MSCs under DMEM-F12 10% FBS, SM-MSC-CM and/or UC-MSC-CM stimuli, at 0 h and after 19, 26 and 43 hours.

FIG. 6: Glycosaminoglycans (GAG) production by (mouse) chondrocytes in vitro as affected by different “cocktail supplement” compositions: SM-MSC-CM alone, UC-MSC-CM alone, or a 1:1 composition of SM-MSC-CM:UC-MSC-CM.

FIG. 7: Qualitative ranking of the soleus distal tendinous insertion regeneration 21 days after partial tenectomy, considering Lesion size, Fibre density, Fibre alignment and Tendon integrity.

FIG. 8: Global Formulation viability for SM-MSC in AS ou UCBP, at thawing (0 h) and after 24, 48 or 72 hours, when maintained at RT or 4° C.

FIG. 9: Detail on the Formulation viability for SM-MSC in AS or UCBP, 72 after thawing hours, when maintained at RT or 4° C.

FIG. 10: Experimental Design for Final Formulation viability assessment of SM-MSC.

### DESCRIPTION OF THE INVENTION

For the purposes of defining the embodiments of the present invention, the path for MSCs utilization in cell therapy will be divided in the following steps 1) Tissue procurement, 2) MSCs isolation, 3) MSCs culture (expansion/multiplication), 4) MSCs freeze/thawing, 5) MSCs formulation, 6) MSCs manipulation, 7) MSCs administration.

It is in the basis of the present invention the assumption that developments can be made in the steps 1) through 7) above to improve the therapeutic performance of the resulting cell-based composition.

Although the present invention can be applied to any type of cell-based composition, in a preferred embodiment of the invention it is applied to compositions using cell populations obeying to the minimal criteria for defining multipotent mesenchymal stromal/stem cells, as defined by the International Society for Cellular Therapy (ISCT) in the 2006 position statement, namely the homogeneous cell population obtained in steps 1) trough 3) above must be plastic-adherent when maintained in standard MSC culture conditions.

Second, cells within the population must express CD105, CD73 and CD90, and must lack the expression of CD45, CD34, CD14 or CD11b, CD79alpha or CD19 and HIA-DR surface molecules. And lastly, cells within the population must be able to undergo in vitro differentiation into osteoblasts, adipocytes and chondrocytes (Dominici et al., 2006).

Although the mesenchymal stromal/stem cell-based compositions resulting from the invention can be applied to many therapeutic applications, in a preferred embodiment, the resulting compositions are used for treatment of musculoskeletal tissues, namely tendon, ligament, cartilage, bone and muscle that have suffered damage, whether from age-related degeneration, continued use-related degeneration, effort-related degeneration, illness-related degeneration, congenital condition-related degeneration, osteoarthritic symptoms in general and accident-related tear out characterized by a stage of acute inflammation.

As such, in a preferred embodiment, the population of cells obtained from steps 1) through 3) above, are derived either from synovial tissues, preferably from the synovial membrane lining the osteochondral tissues of the joint (SM-MSCs), given their chondrogenic lineage and natural role in joint tissue regeneration and homeostasis.

In another embodiment of the invention, the population of cells obtained from steps 1) through 3) above, shall be derived from mammal tissue.

In a preferred embodiment of the invention, the population of cells obtained from steps 1) through 3) above, shall be derived from human tissue.

In a preferred embodiment of the invention, the population of cells obtained from steps 1) through 3) above, shall be derived from canine tissue.

In a preferred embodiment of the invention, the population of cells obtained from steps 1) through 3) above, shall be derived from equine tissue.

In a preferred embodiment of the invention, the population of cells obtained from steps 1) through 3) above, shall be derived from camelid tissue.

In a preferred embodiment of the invention, the population of cells obtained from steps 1) through 3) above, shall be derived from feline tissue.

Supporting the inventive step of the invention is the innovative assumption that by taking advantage of distinct beneficial effects from two different types of MSCs, one can (synergistically) achieve cumulative beneficial results.

Thus, a major embodiment of the invention comprises the use of the best suited MSC type (lineage-specific) to promote tissue regeneration, leveraging a “cocktail supplement” containing a set of paracrine trophic factors that recognizably confer umbilical cord tissue (Wharton's Jelly)-derived MSCs with their paracrine regenerative activity.

In a preferred embodiment of the invention, the cocktail supplement is a defined mixture of cytokines, metabolites and other growth factors, which composition has been drawn, but not limited to, from a judicious analysis of the secretome and metabolome of the umbilical cord blood plasma (UCBP—examples 1.a and 1.b) and in media conditioned by UC-MSCs maintained in self-aggregated spheroids cultured in three-dimensional culture conditions. More specifically, the cocktail supplement contains, respecting the relative proportions,
HGF in an amount of 10-250 pg/mL, preferably of 50-200 pg/mL, more preferably 75-150 pg/mL, even more preferable 100 pg/mL;
EGF in an amount of 10-250 pg/mL, preferably of 15-150 pg/mL, more preferably 25-100 pg/mL, even more preferably of 35-75 pg/mL, preferably of 50 pg/mL;
KGF in an amount of 10-250 pg/mL, preferably of 15-150 pg/mL, more preferably 25-100 pg/mL, even more preferably of 35-75 pg/mL, preferably of 50 pg/mL;
TGFβ-1 in an amount of 0.5-10.0 ng/mL, preferably of 1.0-7.5 ng/mL, more preferably of 2.5-5.0 ng/mL;
TGFβ-2 in an amount of 0.5-10.0 ng/mL, preferably of 0.75-7.5 ng/mL, more preferably of 1.0-2.5 ng/mL;
TGFβ-3 in an amount of 10-250 pg/mL, preferably of 50-200 pg/mL, more preferably of 100-150 pg/mL;
G-CSF in an amount of 10-250 pg/mL, preferably of 25-150 pg/mL, more preferably of 50-100 pg/mL;
VEGF-A in an amount of 10-250 pg/mL, preferably of 25-150 pg/mL, more preferably of 50-100 pg/mL;
FGF2 in an amount of 10-250 pg/mL, preferably 25-150 pg/mL, more preferably of 50-100 pg/mL;
LIF in an amount of 1.0-250 pg/mL, preferably of 10-150, more preferably 50-100 pg/mL;
IL-8 in an amount of 10-250 pg/mL, preferably 20-150 pg/mL, more preferably 40-100 pg/mL;
Eotaxin-1 in an amount of 10-250 pg/mL, preferably of 50-200 pg/mL, more preferably 100-125 pg/mL;
MCP-1 in an amount of 0.5-10.0 ng/mL, preferably of 0.75-7.5, more preferably of 1.0-5.0 ng/mL);
PDGF-BB in an amount of 0.5-10.0 ng/mL, preferably of 1.0-7.0 ng/mL, more preferably 2.5-5.0 ng/mL;
CCL5 in an amount of 0.5-10.0 ng/mL, preferably of 1.0-7.5, ng/mL more preferably of 3.0-5.0 ng/mL; and
sCD40L in an amount of 0.5-10.0 ng/mL, preferably of 1.0-7.5 ng/mL, more preferably of 2.0-40 ng/mL.

In an alternative embodiment of the invention, the “cocktail supplement” comprises umbilical cord blood plasma (UCBP) obtained from umbilical cord blood (UCB) that is collected from the umbilical vein by gravity into a collecting bag containing citrate-phosphate-dextrose (CPD) and transported to the laboratory at refrigerated temperature ranging between 4° C. and 22° C. UCB is processed within 72 hours after collection using an A×P system® (Thermogenesis) to separate the UCBP, erythrocytes and concentrated mononuclear cell (MNC) fractions.

Yet in an alternative embodiment of the invention, the “cocktail supplement” comprises the growth medium conditioned by UC-MSCs maintained in either normal two-dimensional monolayer cultures or preferentially in three-dimensional culture conditions where they self-assemble to form spheroids.

Given that, in a preferred embodiment of the invention, the resulting compositions are to be used in the treatment of musculoskeletal tissues, a preferred embodiment's composition of the invention contains SM-MSCs, leveraged by the “cocktail supplement” specifically in steps 4) MSCs freeze/thawing, 5) MSC formulation and 6) MSCs manipulation.

In an alternative embodiment of the invention, SM-MSC in the final formulation are replaced by growth medium conditioned by the same SM-MSC (SM-MSC-CM), that are maintained in either normal two-dimensional monolayer cultures or preferentially in three-dimensional culture conditions where they self-assemble to form spheroids. In this alternative embodiment, the final formulation consists of a 1:1 mixture of SM-MSC-CM, leveraged by “cocktail supplement” specifically in step 5) MSC formulation.

The specific effects of the “cocktail supplement”, in steps 4) MSCs freeze/thawing, and 5) MSCs formulation and 6) MSCs manipulation, represent developments that would not be obvious to the expert in the area, namely:

In Step 4) MSC freeze/thawing—The use of the “cocktail supplement” either in the freezing solution (90% cocktail:10% DMSO) or in the recovery diluting solution (100% cocktail), increases the number of viable cells recovered upon thawing by 80% or 60%, respectively; when compared to the threshold performance (95% recovery) of the solution most commonly used in the state-of-the-art, containing 90% Foetal Bovine Serum (FBS):10% DMSO (example 2).

In Step 5) MSC formulation—The use of the “cocktail supplement” in the MSCs formulation (cells+100% cocktail, or 1:1 mixture of SM-MSC-CM and “cocktail supplement”):
i. Increases the formulation's motility induction capacity of surrounding synoviocytes (an important mechanism involved in joint tissue healing/regeneration) by 30% when compared to SM-MSC or SM-MSC-CM alone (example 3);
ii. Increases the formulation's capacity to induce glycosaminoglycan (GAG) production by surrounding chondrocytes (essential for new cartilage formation) by 6-fold when compared to SM-MSC or SM-MSC-CM alone (example 4);
iii. Increases the formulation's capacity to reduce lesion size, increase fibre density, improve fibre alignment and tendon integrity in vivo, by 30%, in the first 21 days after partial tenectomy of the soleus distal tendinous insertion, when compared to SM-MSC or SM-MSC-CM alone (example 5).

### In Step 6) MSC Manipulation

iv. Increases cell viability within the formulation with time, at 4° C. and room temperature (RT−22±1° C.), when compared to autologous serum: 48 h at 4° C. by 6%; 72 h at 4° C. by 10%; 48 h at RT by 14% and 72 h at RT by 26% (example 6).

### EXAMPLES

### Example 1

1.a) Human Umbilical Cord Blood Plasma (hUCBP) Secretome Analysis

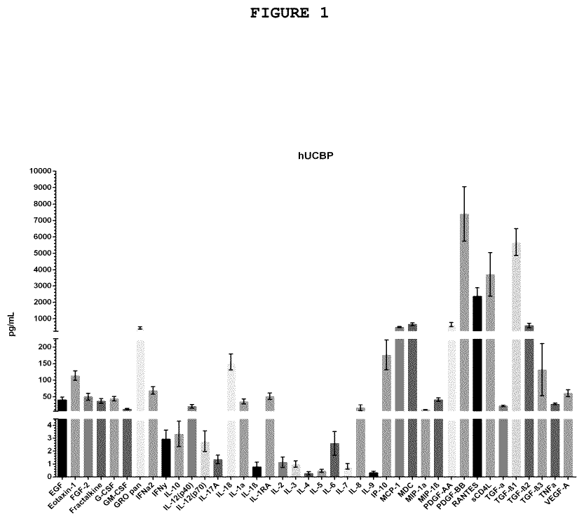

Ref. to FIG. 1: Detection and Quantification in pg/mL of cytokines, chemokines and growth factors, through Multiplexing LASER Bead Analysis (Eve Technologies, Calgary, Alberta, Canada) in hUCBP presented as Mean±SE.

### Materials and Methods:

UCBP Collection and Preparation: Maternal and neonatal pairs were evaluated during the antenatal period in the maternity wards at different hospitals. Upon delivery, and following informed consent, hUCB was collected from the umbilical vein by gravity into a 150 mL volume bag containing 21 ml of citrate-phosphate-dextrose (CPD). This procedure was performed by trained midwives after delivery of the placenta. The hUCB was stored at 4° C.±2° C. until processing for cryopreservation. Human UCB samples were transported to the laboratory at refrigerated temperatures ranging between 4° C. and 22° C., within 72 hours after collection. The A×P system® (Thermogenesis) was used to separate the whole blood into three layers of red blood cells (RBC), concentrated mononuclear cell (MNC) and hUCBP bag and a freezing bag.

Quantification and Detection of Bioactive Molecules: Plasma samples were collected, aliquoted and stored for the Detection and Quantification of cytokines, chemokines and growth factors, through Multiplexing LASER Bead Analysis (Eve Technologies, Calgary, Alberta, Canada).

Selected panels included: epidermal growth factor (EGF), eotaxin-1, fibroblast growth factor 2 (FGF-2), fms-related tyrosine kinase 3 ligand (Flt-3L), fractalkine, granulocyte colony-stimulating factor (G-CSF), granulocyte macrophage colony-stimulating factor (GM-CSF), GRO(pan), interferon-alpha 2 (IFNα2), interferon-gama (IFNγ), several interleukins (IL-la, IL-1l, IL-ira, IL-2, IL-3, IL-4, IL-5, IL-6, IL-7, IL-8, IL-9, IL-10, IL-12 (p40), IL-12 (p70), IL-13, IL-15, IL-17A), interferon gama-induced protein 10 (IP-10), monocyte chemotactic protein-1 (MCP-1), monocyte chemotactic protein-3 (MCP-3), macrophage-derived chemokine (MDC), macrophage inflammatory protein-1 alpha (MIP-la), macrophage inflammatory protein-1 beta (MIP-1l), platelet-derived growth factor—AA (PDGF-AA),), platelet-derived growth factor—AB/BB(PDGF-AB/BB), chemokine (C—C motif) ligand 5 (RANTES or CCL5), soluble CD40 ligand (sCD40L), transforming growth factor alpha (TGFα), tumor necrosis factor alpha (TNFα), tumor necrosis factor beta (TNFβ), vascular endothelial growth factor A (VEGF-A) [Human Primary Cytokine Array/Chemokine Array 41-Plex Panel], and tumor growth factor beta 1, 2 and 3 (TGF-β 1, 2, and 3) [TGF-b 3-Plex Array Multi-Species].

The multiplex assay was performed at Eve Technologies by using the Bio-Plex™ 200 system (Bio-Rad Laboratories, Inc., Hercules, CA, USA), and a Milliplex human cytokine kit (Millipore, St. Charles, MO, USA) according to their protocol.

The assay sensitivities of these markers range from 0.1-10.1 pg/mL. The 3 TGF-β isoforms were simultaneously quantified through the Discovery Assay® TGFβ3-Plex Cytokine Array (Eve Technologies Corp, Calgary, Alberta, Canada).

Bio-Plex™ 200 system (Bio-Rad Laboratories, Inc., Hercules, CA, USA) was also employed, and a Milliplex TGF-β 3-plex kit (Millipore, St. Charles, MO, USA) according to their protocol. The assay sensitivities of these markers range from 2.2-6.6 pg/mL. Finally, using the Bio-Plex™ 200 system (Bio-Rad Laboratories, Inc., Hercules, CA, USA), and a Milliplex Human Angiogenesis/Growth Factor kit (Millipore, St. Charles, MO, USA) according to their protocol. The assay's sensitivity for leptin is approximately 42.8 pg/mL. The sensitivities of the other markers range from 0.2-17.0 pg/mL. For all of the three arrays, individual analyte values and other assay details are available on Eve Technologies' website or in the Milliplex protocol.

Results were plotted using GraphPad Prism version 6.00 for Mac OS X, GraphPad Software, La Jolla California USA, and presented as Mean±Standard Error of the Mean (SE) (FIG. 1).

1.b) Human Umbilical Cord Blood Plasma (hUCBP) Metabolome Analysis

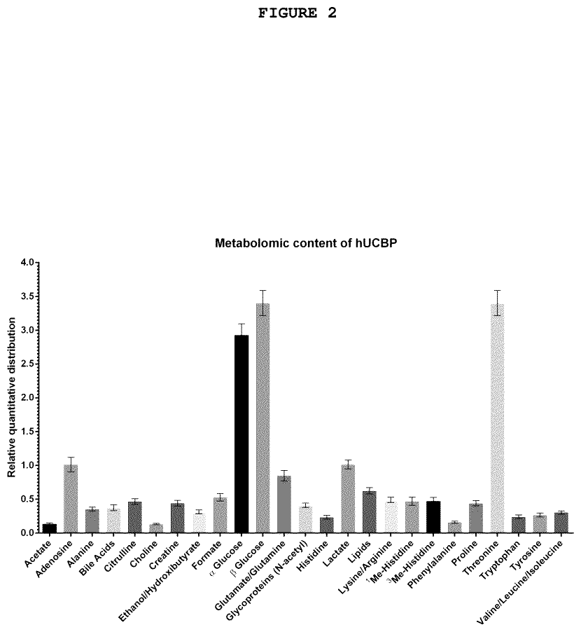

Refer to FIG. 2: Relative quantitative distribution of the metabolites in hUCBP presented as Mean±SE.

TABLE 1
Relative quantitative distribution of metabolites
observed in the 1H-NMR spectra of hUCBP

| | | | Chem. | |
|---|---|---|---|---|
| | | | Shifts | hUCBP |
| Metabolites | Code | Group | (ppm) | (N = 13) |
| 1Methylhistidine | 1Me-His | CH | 7.8 | 0.47 ± 0.23 |
| 3Methylhistidine | 3Me-His | H2 | 7.61 | 0.47 ± 0.21 |
| Acetate | Ace | CH3 | 1.92 | 0.14 ± 0.05 |
| Adenosine | ADP/ATP | CH | 8.22 | 1.01 ± 0.41 |
| Alanine | Ala | CH3 | 1.48 | 0.35 ± 0.12 |
| Bile Acids | BA | CH3 | 0.5-0.8 | 0.37 ± 0.16 |
| Choline | Cho | NCH3 | 3.21-3.24 | 0.13 ± 0.11 |
| Citrulline | Citr | CH2 | 3.12 | 0.47 ± 0.16 |
| Creatine | Cr | CH3 | 3.04 | 0.44 ± 0.16 |
| Ethanol/ | EtOH/HB | CH3 | 1.21 | 0.31 ± 0.11 |
| Hydroxybutyrate | | | | |
| Formate | For | CH | 8.58 | 0.53 ± 0.21 |
| Glutamate/ | Glu/Gln | CH2 | 2.34 | 0.85 ± 0.30 |
| Glutamine | | | | |
| Glycoproteins | NAG | CH3 | 2.04 | 0.41 ± 0.13 |
| (N-acetyl) | | | | |
| Histadine | His | H2 | 7.73 | 0.23 ± 0.11 |
| Lactate | Lac | CH | 4.14 | 1.01 ± 0.25 |
| Lipids | Lip | CH3 | 0.8-0.9 | 0.62 ± 0.18 |
| Lysine/Arginine | Lys/Arg | CH/CH2 | 1.6-1.7 | 0.49 ± 0.15 |
| Phenylalanine | PheAla | H3.5 | 7.44 | 0.16 ± 0.07 |
| Prolina | Pro | H2 | 4.2 | 0.44 ± 0.16 |
| Threonine | Thr | CH3 | 1.33 | 0.32 ± 0.13 |
| Tryptophan | Try | H7 | 7.56 | 0.24 ± 0.11 |
| Tyrosine | Tyr | H3.6 | 7.56 | 0.27 ± 0.11 |
| Valine/Leucine/ | Val/Leu/Ile | CH3 | 0.9-1.1 | 0.30 ± 0.09 |
| Isoleucine | | | | |
| α-Glucose | α-Glu | CH (H1) | 5.24 | 2.93 ± 0.63 |
| β-Glucose | β-Glu | CH (H1) | 4.65 | 3.40 ± 0.70 |

### Materials and Methods:

UCBP Collection and Preparation: Maternal and neonatal pairs were evaluated during the antenatal period in the maternity wards at different hospitals. Upon delivery, and following informed consent, hUCB was collected from the umbilical vein by gravity into a 150 mL volume bag containing 21 ml of citrate-phosphate-dextrose (CPD). This procedure was performed by trained midwives after delivery of the placenta. The hUCB was stored at 4° C.±2° C. until processing for cryopreservation. Human UCB samples were transported to the laboratory at refrigerated temperatures ranging between 4° C. and 22′C, within 72 hours after collection. The A×P system® (Thermogenesis) was used to separate the whole blood into three layers of red blood cells (RBC), concentrated mononuclear cell (MNC) and hUCBP bag and a freezing bag. Plasma samples were collected, aliquoted and stored for NMR Spectroscopy analysis.

Metabolomic Analysis: 1H-NMR spectra of all hUCBP and FBS samples were acquired with water suppression using 1D NOESY and CPMG pulse sequences. The implementation of 1D NOESY has led to 1H NMR spectra with improved solvent peak (at 4.70 ppm) suppression but complex line shapes and highly overlapped resonances from the low molecular weight metabolites and macromolecular components. To reduce the complexity of the spectra, T2-edited CPMG experiments with appropriate T2 delays were used to attenuate the broad signals from high molecular weight species and facilitate the identification of small molecular components. The average 1H (CPMG) NMR spectra of hUCBP samples is shown in FIG. 2. The assignment of the resonance signals in the spectra was based on results obtained from various 1D and 2D (1H/1H COSY, 1H/1H TOCSY, 1H/13C HSQC) NMR experiments and reference data. The analysis of 1H-NMR spectra has allowed the identification and quantification of a number of metabolites in hUCBP listed in Table 1. Results were plotted using GraphPad Prism version 6.00 for Mac OS X, GraphPad Software, La Jolla California USA, and presented as Mean±Standard Error of the Mean (SE).

### Example 2) MSC Freeze/Thawing

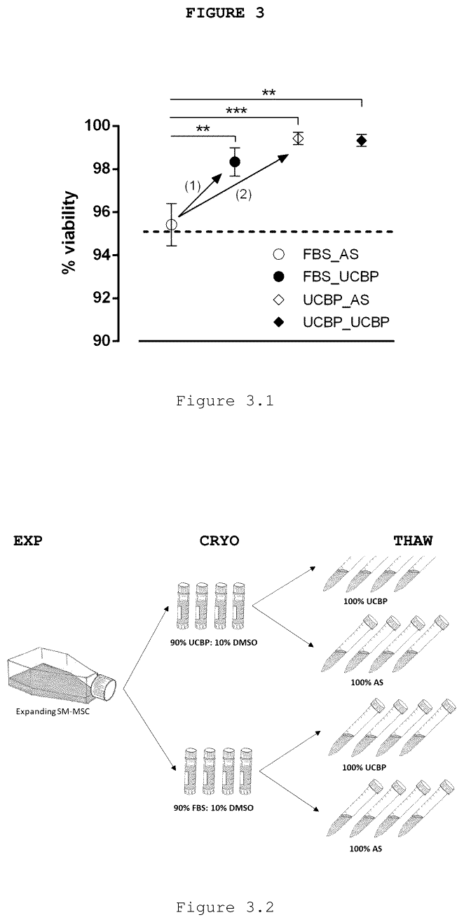

FIG. 3.1: SM-MSC viability upon thawing using either AS or UCBP, after cryopreservation in 90% FBS or UCBP: 10% DMSO. Significant differences are indicated according to P values with one, two, three or four of the symbols (*) corresponding to 0.01<p≤0.05, 0.001<p≤0.01, 0.0001<p≤0.001 e p<0.0001, respectively.

### Materials and Methods:

Freeze/Thawing of SM-MSC. The “cocktail supplement” (90% umbilical cord blood plasma: 10% DMSO): UCBP was used either in the freezing solution, and compared to the currently used 90% Foetal Bovine Serum (FBS):10% DMSO: FBS; or in the thawing solution (100′ umbilical cord blood plasma): UCBP versus autologous serum: AS. When UCBP is used in the thawing solution, with cell frozen in FBS, the number of viable cells recovered upon thawing is 60% higher than when AS is used for thawing-arrows (1). In turn, when UCBP is used in the freezing solution, and cells are thawed in AS, the number of viable cells recovered upon thawing is 80% higher than when FBS is used for freezing—arrow (2). % differences are calculated with respect to the threshold performance (95k recovery) of the conditions most commonly used in the state-of-the-art: MSC-FBS-AS.

### Materials and Methods:

Cell isolation: SM-MSCs are isolated from fresh tissue from young and healthy animals obtained through synovial membrane biopsies from certified slaughter houses, transported to the laboratory facilities in a hermetically sealed sterile container in saline buffer with antibiotics, and processed within a period up to 48 h. The synovial tissue is digested using collagenase and the isolated cells are incubated in a static monolayer culture using standard MSCs basal medium supplemented with 20% FBS, at 39′=C, in 7% CO₂ humidified atmosphere until they reach confluence. When needed, cells from confluent cultures are cryopreserved in 10% dimethylsulphoxide (DMSO) and FBS, at a concentration of 3×106 cells/ml, using control rate temperature decrease (CRF).

UCBP Collection and Preparation: Maternal and neonatal pairs were evaluated during the antenatal period in the maternity wards at different hospitals. Upon delivery, and following informed consent, hUCB was collected from the umbilical vein by gravity into a 150 mL volume bag containing 21 ml of citrate-phosphate-dextrose (CPD). This procedure was performed by trained midwives after delivery of the placenta. The hUCB was stored at 4° C.±2° C. until processing for cryopreservation. Human UCB samples were transported to the laboratory at refrigerated temperatures ranging between 4° C. and 22° C., within 72 hours after collection. The A×P system® (Thermogenesis) was used to separate the whole blood into three layers of red blood cells (RBC), concentrated mononuclear cell (MNC) and hUCBP bag and a freezing bag.

Plasma samples were collected, aliquoted and Heat-inactivated (56° C. in water bath, for 30 minutes with gentle agitation) to prevent coagulation when added to cellular systems (step required in addition to the CPD anticoagulant in collection bath).

Freeze-Thaw viability assessment: SM-MSC in expansion (in CMEM supplemented with 20% FBS, at 37° C. and 5% CO2 humidified atmosphere) were harvested with resource to Trypsin (Trypsin-EDTA 0.25%) and cryopreserved in either 90% FBS: 10% DMSO or 90% UCBP: 10′ DMSO, at a 2, 5×105 cells/mL. Cryovials were placed in gradual freezing containers, at 80° C. overnight, and transferred to liquid phase nitrogen storage units (−195.79° C.). After 3 weeks, cryovials were selected from the cryogenic storage unit and thawed.

Vials were carefully placed in a water bath (37° C.) and allowed to thaw partially. Then, 500 μL of UCBP (or FBS) was added to the cryovial and the cell suspension was transferred to a centrifuge tube. The cryovial was additionally washed with 2500 μL of UCBP (or FBS), and the suspension added to the same centrifuge tube. DPBS (6 mL) was added for a final volume of 10 mL of cellular suspension. Cell suspension was centrifuged at 200 gx for 10 minutes and supernatant was discarded.

Cell pellet was resuspended in 1 mL of UCBP (or FBS) and cell viability was assessed using Trypan Blue exclusion dye assay (10 μL Trypan Blue: 10 μL of cell suspension). Triplicate counting was performed using an automated cell counter. Statistical analysis was performed using the GraphPad Prism version 6.00 for Mac OS X, GraphPad Software, La Jolla California USA. The experiments were performed in 4 replicates and the results were presented as Mean±Standard Error of the Mean (SE). Analysis was performed by one-way ANOVA test followed by Brown-Forsythe test. Differences were considered statistically significant in a Confidence Interval (CI) of 95%. Results significance are presented as compared to control sample and represented with the symbol (*) and compared to FBS_AS, and represented by the symbol (#). Significance results are also indicated according to P values with one, two, three or four of the symbols (*) corresponding to 0.01<p≤0.05, 0.001<p≤50.01, 0.0001<p≤0.001 e p<0.0001, respectively.

### Experimental Design (Scheme 1)

Refer to FIG. 3.2: Experimental design for the Freeze-Thaw viability assessment of SM-MSCs.

### Example 3) Synoviocyte Motility Induction

In vitro scratch assays of equine synoviocytes. The “cocktail supplement” was added to confluent monolayer cultures of equine synoviocytes where a scratch has been made to reproduce a wound. The effect of the “cocktail supplement” (100% growth medium conditioned by equine UC-MSC maintained in self-assembled spheroids in three-dimensional culture conditions, 3× concentrated) alone: UC-MSC-CM on the capacity of synoviocytes to migrate towards the open space and close the scratched area, was compared to that of growth medium conditioned by equine SM-MSC in the same conditions, alone: SM-MSC-CM and with a 1:1 mixture of both conditioned media:SM-MSC-CM:UC-MSC-CM to evaluate if there was a cumulative effect on synoviocyte motility caused by SM-MSC:UC-MSC paracrine synergies. As a control, rich synoviocyte culture medium was also used in parallel: DMEM-F12 10% FBS. The figure shows 30% increase in synoviocyte motility, as measured by integrating the scratch area 43 h after scratch induction meaning that there is a clear cumulative effect resulting from synergistic activities between SM-MSC and UC-MSC.

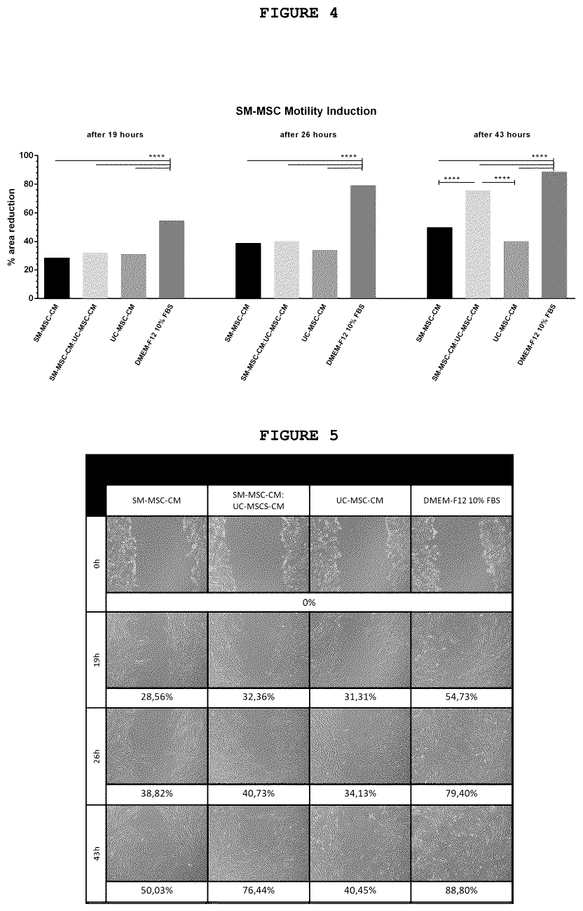

Refer to FIG. 4: Percentage of reduction in the scratched area of SM-MSCs under DMEM-F12 10% FBS, SM-MSC-CM and/or UC-MSC-CM stimuli, after 19, 26 and 43 hours. Statistical differences were considered at P<0.05, and significance of the results is indicated according to P values with one, two, three or four of the symbols (*) corresponding to 0.01≤P<0.05; 0.001≤P<0.01; 0.0001≤P<0.001 and P<0.0001, respectively.

Refer to FIG. 5: Microphotographs and % of area reduction of the scratched area of SM-MSCs under DMEM-F12 10% FBS, SM-MSC-CM and/or UC-MSC-CM stimuli, at 0 h and after 19, 26 and 43 hours.

### Materials and Methods:

Conditioning Protocol: For the production of Conditioned Media (CM), two equine derived cell populations were explored: Synovial Membrane Mesenchymal Stem Cells (SM-MSCs) and Umbilical Cord Stroma/Wharton's jelly Mesenchymal Stem Cells (UC-MSCs).

SM and UC-MSCs were thawed and expanded in standard culture conditions in a T75 flask (initial seeding of 6.000 to 8.000 cells/cm2), using cultured with MEM-α+GlutaMAX™ supplemented with FBS (20% v/v), HEPES buffer (1% v/v), penicillin (100 U/mL)—streptomycin (100 pg/mL), and amphotericin B (2,5 μg/mL). Cells were maintained at 37° C. in a 5% CO2 humidified atmosphere until approximately 80% confluence was reached. Then, the cultures were washed to remove any trace of FBS and other supplements. Washing procedures implies three gentle washes with warm DPBS, followed by two washes with plain DMEM/F12+GlutaMAX™. Finally, cells are placed in plain DMEM/F12+GlutaMAX™ and cultured for 48 hours. After 48 hours of conditioning, culture medium was collected and centrifuged (900×g).

For the preparation of the assay, CM was concentrated five times, using Pierce™ Protein Concentrators (PES, 3K MWCO, Thermo Fisher reference #88514).

Migration assay: SM-MSCs were thawed and in vitro expanded using standard culture protocols. Culture was maintained at 37° C. and 95% humidified atmosphere with 5% CO2 environment. Cells were maintained in αMEM with GlutaMAX™ supplemented with 20% (v/v) FBS, 100 IU/ml penicillin, 0.1 mg/ml streptomycin, 2.05 μg/ml amphotericin B and 10 mM HEPES Buffer solution. Upon 80% confluency, cells were harvested using Trypsin (Trypsin-EDTA (0.25%)) and plated at 10.000 cells/cm2 in a 24 well-plate. Cells were further allowed to expand up to 90% confluency and the centre of the well scratched using a P200 sterile pipette tip.

Wells were individually photographed, and standard culture medium was replaced by defined experimental conditions and appropriate controls:
SM-MSC-CM: 100% SM-MSC-CM concentrated 5×
SM-MSC-CM:UC-MSC-CM: 50% SM-MSC-CM concentrated 5×+50% UC-MSCs-CM concentrated 5×
UC-MSC-CM: 100% UC-MSC-CM concentrated 5×
DMEM-F12 10% FBS: supplemented with 10% v/v FBS

Cells were cultured in the conditions above described and observed for proliferation/migration into the scratched area. Microphotographs (Zeiss Axiovert® 40 CFL) were taken at 19, 26 and 43 hours after experimental media addition and media was changed after each procedure. Photographs were obtained from marked areas along each scratch line, allowing for the monitoring of cell response in multiple areas.

Photographs were then analysed using the ImageJ Software (ImageJ 1.51k, NIH, USA) (FIG. 5) and the scratched area were measured in time sequenced images. Data was then plotted and treated using GraphPad Prism version 6.00 (GraphPad Software, La Jolla California USA) (FIG. 4).

Six measurements per condition were considered and the results were presented as ‘scratched area (pixels)’ and ‘percentage reduction in scratched area’. Data was further analyzed for significant differences. Multiple comparisons test was performed by one-way ANOVA supplemented with Tukey's HSD post-hoc test. Differences were considered statistically significant at P<0.05. Significance of the results is indicated according to P values with one, two, three or four of the symbols (*) corresponding to 0.01≤P<0.05; 0.001≤P<0.01; 0.0001≤P<0.001 and P<0.0001, respectively.

### Example 4) GAG Production by Chondrocytes

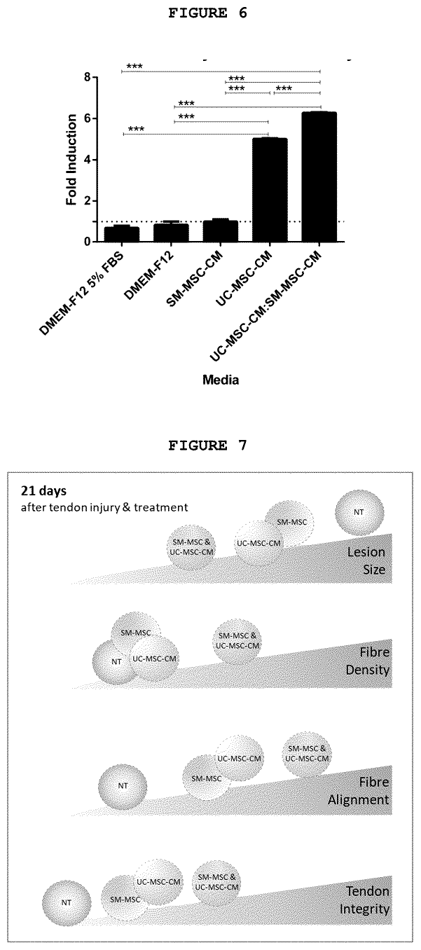

Refer to FIG. 6: Glycosaminoglycans (GAG) production by (mouse) chondrocytes in vitro as affected by different “cocktail supplement” compositions: SM-MSC-CM alone, UC-MSC-CM alone, or a 1:1 composition of SM-MSC-CM:UC-MSC-CM.

Quantification of glycosaminoglycans (GAG) produced by (mouse) chondrocytes in vitro. Different formulations were added to confluent monolayer cultures of mouse chondrocytes (ATDC5) to evaluate in which conditions GAG production—one of the most relevant mechanisms involved in new (healthy) cartilage formation—would be induced. The effect of the “cocktail supplement” (100% growth medium conditioned by equine UC-MSC in conventional monolayer culture conditions, 5× concentrated) alone: UC-MSC-CM, was compared to that of growth medium conditioned by equine SM-MSC in the same conditions, alone: SM-MSC-CM and with a 1:1 mixture of SM-MSC-CM and “cocktail supplement”: SM-MSC-CM:UC-MSC-CM to confirm:
1) the higher paracrine activity of UC-MSC over SM-MSC, and
2) if there was a cumulative effect on induced GAG production by SM-MSC:UC-MSC paracrine synergies.

As controls, chondrocyte culture medium (DMEM-F12, 5% FBS) and CM solvent (DMEM-F12), were used as controls. The figure shows 1) a 5-fold higher paracrine activity of UC-MSC over SM-MSC to promote chondrocyte GAG induction and 2) a 6-fold higher paracrine activity of the 1:1 mixture of both conditioned media, effect resulting from synergistic activities between SM-MSC and UC-MSC. The effects of the different media were evaluated by one-way ANOVA with Tukey's test. *, **, *** Significantly differs from the different media composition and the days of differentiation with P<0.05, P<0.01 and P<0.001, respectively. #, ##, ###GAG production significantly induced with P<0.05, P<0.01 and P<0.001, respectively.

### Materials and Methods:

Mouse chondrocyte (ATDC5 cell line) culture: Mouse chondrocytes (ATDC5 cell line) were seeded at 1×104 cells/cm2 and cultured in DMEM-F12 supplemented with 5% FBS, at 37° C., in 5% CO2 humidified atmosphere until they reach 70-80% confluence. Cell passage was performed by Trypsin/EDTA 0.25% incubation for 5 minutes at 37° C., approximately every 72 h.

GAG quantification: GAGs were quantified in ATDC5 cells seeded in 96-well plates. At a confluence of 30 to 40%, cells were incubated with UC-MSC-CM, SM-MSC-CM, SM-MSC-CM:UC-MSC-CM and as controls, DMEM-F12 and DMEM-F12 with 5% FBS. GAGs were quantified at 48 h post-incubation using the Blyscan™ Sulfated Glycosaminoglycan Assay from Biocolor (Carrickfergus, UK), according to the manufacturer's instructions. The Blyscan Assay is a quantitative dye-binding method for the analysis of sulphated proteoglycans into tissue culture medium and extracted from biological materials, namely soluble chondroitin, keratan, dermatan and heparan sulfates and insoluble GAG (following solubilization by papain). A total of three independent experiments were performed.

### Example 5) Tendon Healing Capacity In Vivo

Refer to FIG. 7: Qualitative ranking of the soleus distal tendinous insertion regeneration 21 days after partial tenectomy, considering Lesion size, Fibre density, Fibre alignment and Tendon integrity.

Treatment of tendon lesions in vivo using a rat partial tenectomy of the soleus distal tendinous insertion model. Different formulations were used to treat injured tendons to demonstrate the beneficial cumulative effects brought by the “cocktail supplement” (100% growth medium conditioned by equine UC-MSC maintained in self-assembled spheroids in three-dimensional culture conditions, 3× concentrated): UC-MSC-CM.

A formulation containing equine SM-MSCS alone: SM-MSCs—was used in one experimental group and its efficacy compared with the same formulation, leveraged by the “cocktail supplement”, UC-MSC-CM, specifically in steps 4) MSC freeze/thawing, 5) MSC Formulation and 6) MSC manipulation SM-MSC & UC-MSC-CM. A group containing the “cocktail supplement” alone: UC-MSC-CM, and a group of untreated lesions (NT) were used as controls.

A scoring from 0-100% (left-to-right) was used to measure several parameters as depicted in the figure.

In summary, the formulation leveraged by the “cocktail supplement” has reduced lesion size, increase fibre density, improve fibre alignment and augmented tendon integrity in vivo, on AVG by 30% (confirm) when compared to SM-MSC alone, 21 days after lesion and infliction and treatment administration (partial tenectomy of the soleus distal tendinous insertion).

Noticeable was the performance of UC-MSC-CM alone which was as efficient as SM-MSC alone is this model system (FIG. 7).

### Materials and Methods:

UC-MSC-CM preparation in 3D cultures: Using a dynamic culture system (spinner vessels with ball impeller) with αMEM supplemented with 15% FBS, UC-MSC were inoculated with single cell suspensions obtained from two-dimensional cultures at a concentration of 1×106 cells/mL. To promote cell aggregation, the spinner vessels are agitated at 80 rpm and kept at 37° C. in a humidified atmosphere of 5% CO2. After 24 h, FBS concentration was reduced to 5%. After 3 days, medium was replaced by MSC medium without FBS which is allowed to be in contact with the spheroids during 48 h, After the conditioning period, culture medium was collected and centrifuged (900×g), and further concentrated 3 times, using Pierce™ Protein Concentrators (PES, 3K MWCO, Thermo Fisher reference #88514). A volume of 100 μL of the 3× concentrated UC-MSC-CM was injected directly in the area and periphery of the induced tendon injury.

SM-MSC Expansion: SM-MSCs were thawed and in vitro expanded using standard culture protocols. Culture was maintained at 37° C. and 95% humidified atmosphere with 5% CO2 environment. Cells were maintained in αMEM with GlutaMAX™ supplemented with 20% (v/v) FBS, 100 IU/ml penicillin, 0.1 mg/ml streptomycin, 2.05 μg/ml amphotericin B and 10 mM HEPES Buffer solution. Upon 80% confluency, cells were harvested using Trypsin (Trypsin-EDTA (0.25%)), counted, and re-suspended in serum free media. Cells we centrifuged e resulting cell pellets were used for the in vivo assay, at 5×10⁵ SM-MSC per lesion.

Tendon Injury In vivo regeneration: All the animal testing procedures were in conformity with the Directive 2010/63/EU of the European Parliament and the Portuguese DL 113/2013. Humane end points were followed in accordance to the OECD Guidance Document on the Recognition, Assessment and Use of Clinical Signs as Humane Endpoints for Experimental Animals Used in Safety Evaluation (2000), and adequate measures were taken to minimize pain and discomfort.

Adult male Sasco Sprague Dawley rats (Charles River Laboratories, Barcelona, Spain) weighing 250-300 g, were selected. Animals were maintained in a temperature and humidity controlled room with 12-12 h light/dark cycles, and were allowed normal cage activities under standard laboratory conditions. The animals were fed with standard chow and water ad libitum. Animals were housed for two weeks prior to surgery for acclimatization. For the surgical induction of the partial tenectomy of the soleus distal tendinous insertion, an intraperitoneal (IP) injection delivered a pre-mixed anaesthetic solution [ketamine, 9 mg/100 g body weight (bw), and xylazine, 1.25 mg/100 g bw]. Hair from the caudal limbs was clipped and the skin scrubbed routinely with an iodopovidone 10% solution (Betadine®).

A 5-10 mm long linear incision, was performed in the lateral aspect of the caudal part of the limb, along the gastrocnemious path to the distal calcaneous insertion. Tendinous structures were bluntly dissected the distal insertion of the soleus muscle was isolated. On the lateral aspect of the tendon, a 1 mm2 area was clipped and excised, causing the partial tenectomy of the tendon, providing fibre discontinuity and tissue loss. Animals were assigned to receive treatment with either SM-MSC [5×10⁵] and/or UC-MSC-CM [100 μL of 3× CM]. A control group of untreated lesions (NT) was also included.

Skin was sutured, and animals monitored though the recovery period from anaesthesia. After 21 days of recovery and regeneration, animals were placed under deep anaesthesia and sacrificed by lethal intracardiac injection of 5% sodium pentobarbital.

The intervened tendons collected and fixed in a container with 4% formaldehyde solution for histopathological evaluation. Thorough necropsy was performed to assess for systemic effects of the therapeutic systems, and internal organs collected for histopathological analysis.

Tendons were processed for routine analysis (Haematoxylin-Eosin stain) as well as for special staining techniques for the specific evaluation of collagen content and features (Masson's Trichrome and Picrosirius Red). Samples were blindly evaluated by experienced Veterinary Pathologist and qualitatively ranked according to ‘Lesion Size’, regenerative site ‘Fibre Density’ and ‘Fibre Alignment’, and lesion's boundary ‘Tendon Integrity’ (FIG. 7).

### Example 6) Cell Stability in the Formulation at 4° C. and Room Temperature (RT−22° C.)

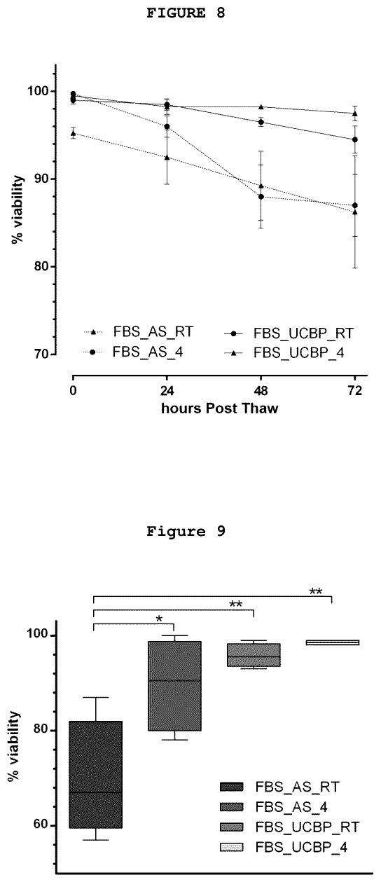

Refer to FIG. 8: Global Formulation viability for SM-MSC in AS or UCBP, at thawing (0 h) and after 24, 48 or 72 hours, when maintained at RT or 4° C.

Refer to FIG. 9: Detail on the Formulation viability for SM-MSC in AS or UCBP, 72 after thawing hours, when maintained at RT or 4° C.

Stability of canine SM-MSC (SM-MSC) after thawing at 4° C. (4) and Room Temperature (RT−22° C.). SM-MSC were frozen in 90% Foetal Bovine Serum (FBS):10% DMSO: FBS90 and then thawed and maintained in either autologous serum: AS or “cocktail supplement” (100% umbilical cord blood plasma): UCBP, at either 4° C. or RT. As it can be seen from in the figure above, the “cocktail supplement” increases SM-MSC viability within the formulation with time, at both 4° C. and RT, when compared to autologous serum: 48 h at 4° C. by 6%; 72 h at 4° C. by 10%; 48 h at RT by 14% and 72 h at RT by 26%.

### Materials and Methods:

UCBP Collection and Preparation: Maternal and neonatal pairs were evaluated during the antenatal period in the maternity wards at different hospitals. Upon delivery, and following informed consent, hUCB was collected from the umbilical vein by gravity into a 150 mL volume bag containing 21 ml of citrate-phosphate-dextrose (CPD). This procedure was performed by trained midwives after delivery of the placenta. The hUCB was stored at 4° C.±2° C. until processing for cryopreservation. Human UCB samples were transported to the laboratory at refrigerated temperatures ranging between 4° C. and 22° C., within 72 hours after collection. The A×P system® (Thermogenesis) was used to separate the whole blood into three layers of red blood cells (RBC), concentrated mononuclear cell (MNC) and hUCBP bag and a freezing bag.

Plasma samples were collected, aliquoted and Heat-inactivated (56° C. in water bath, for 30 minutes with gentle agitation) to prevent coagulation when added to cellular systems (step required in addition to the CPD anticoagulant in collection bath).

Final Formulation viability assessment: SM-MSC in expansion (in αMEM supplemented with 20% FBS, at 37° C. and 5% CO2 humidified atmosphere) were harvested with resource to Trypsin (Trypsin-EDTA 0.25%) and cryopreserved in 90% FBS: 10% DMSO, at a 2,5×105 cells/mL.

Cryovials were placed in gradual freezing containers, at 80° C. overnight, and transferred to liquid phase nitrogen storage units (−195.79° C.). After 3 weeks, cryovials were selected from the cryogenic storage unit and thawed.

Vials were carefully placed in a water bath (37° C.) and allowed to thaw partially. Then, 500 μL of UCBP or FBS was added to the cryovial and the cell suspension was transferred to a centrifuge tube. The cryovial was additionally washed with 2500 μL of UCBP (or FBS), and the suspension added to the same centrifuge tube. DPBS (6 mL) was added for a final volume of 10 mL of cellular suspension.

Cell suspension was centrifuged at 200 gx for 10 minutes and supernatant was discarded. Cell pellet was resuspended in 1 mL of UCBP (or FBS) and cell viability was assessed using Trypan Blue exclusion dye assay (10 μL Trypan Blue: 10 μL of cell suspension). Triplicate counting was performed using an automated cell counter.

Following initial cell viability record, cell suspension was transferred into sealed glass vials containers and maintained at either Room Temperature (RT−22±1° C.) or refrigerated (4° C.). Cell viability of the final formulation was further assessed after 24, 46, and 72 hours for each maintenance condition.

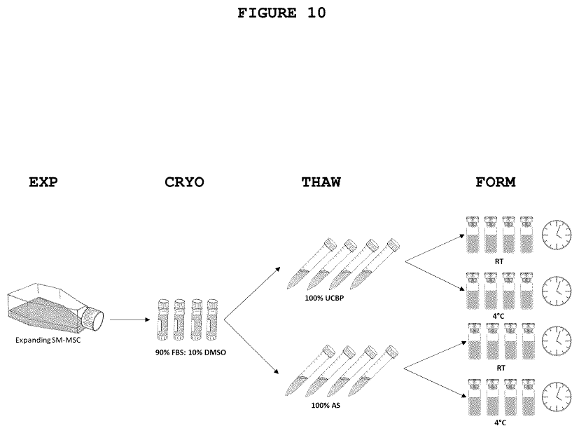

Refer to FIG. 10. Experimental Design for Final Formulation viability assessment of SM-MSC.

Statistical analysis was performed using the GraphPad Prism version 6.00 for Mac OS X, GraphPad Software, La Jolla California USA. The experiments were performed in 4 replicates and the results were presented as Mean±Standard Error of the Mean (SE) (FIGS. 8 & 9). Analysis was performed by one-way ANOVA test followed by Brown-Forsythe test. Differences were considered statistically significant in a Confidence Interval (CI) of 95%. Results significance are presented as compared to control sample and represented with the symbol (*) and compared to FBS_AS, and represented by the symbol (#). Significance results are also indicated according to P values with one, two, three or four of the symbols (*) corresponding to 0.01<P≤0.05, 0.001<P≤0.01, 0.0001<P≤0.001, and P<0.0001, respectively.

### BIBLIOGRAPHIC REFERENCES

Akgun et al. (2015) Matrix-induced autologous mesenchymal stem cell implantation versus matrix-induced autologous chondrocyte implantation in the treatment of chondral defects of the knee: a 2-year randomized study. Arch Orthop. Trauma Surg. 135(2):251-63.
Bárcia et al (2015) What Makes Umbilical Cord Tissue-Derived Mesenchymal Stromal Cells Superior Immunomodulators When Compared to Bone Marrow Derived Mesenchymal Stromal Cells?Stem Cells International, Vol. 2015, Art. ID 583984, 14 pages, http://dx.doi.org/10.1155/2015/583984.
Conconi et al. (2006) CD105(+) cells from Wharton's jelly show in vitro and in vivo myogenic differentiative potential.” International Journal of Molecular Medicine 18(6): 1089-1096.
De Bari et al. (2003) Skeletal muscle repair by adult human mesenchymal stem cells from synovial membrane. The Journal of Cell Biology 160(6): 909-918.
De la Garza-Rodea et al. (2012). “Myogenic properties of human mesenchymal stem cells derived from three different sources.” Cell Transplantation 21(1): 153-173.
Dominici et al. (2006) Minimal criteria for defining multipotent mesenchymal stromal cells. The International Society for Cellular Therapy position statement. Cytotherapy, 8(4):315-7.
Iwai et al. (2010) Ex vivo cartilage defect model for the evaluation of cartilage regeneration using mesenchymal stem cells J Biosci. Bioeng. 111(3):357-64.
Goldberg et al. (2017) The use of mesenchymal stem cells for cartilage repair and regeneration: a systematic review. J Orthop Surg Res. 12: 39.
Ju et al. (2008) Synovial mesenchymal stem cells accelerate early remodelling of tendon-bone healing. Cell Tissue Res. 332:469-478.
Lee et al. (2011) Synovium-derived mesenchymal stem cells encapsulated in a novel injectable gel can repair osteochondral defects in a rabbit model. Tissue Eng Part A. 18(19-20):2173-86.
Lee et al. (2013) Synovial membrane-derived mesenchymal stem cells supported by platelet-rich plasma can repair osteochondral defects in a rabbit model. Arthroscopy. 29 (6):1034-46.
Miranda et al. (2015) The Human Umbilical Cord Tissue-Derived MSC Population UCX® Promotes Early Motogenic Effects on Keratinocytes and Fibroblasts and G-CSF-Mediated Mobilization of BM-MSCs When Transplanted In Vivo. Cell Transplant 24(5):865-77.
Nakamura et al. (2012) Arthroscopic, histological and MRI analyses of cartilage repair after a minimally invasive method of transplantation of allogeneic synovial mesenchymal stromal cells into cartilage defects in pigs. Cytotherapy. 2012; 14(3):327-38.
Nascimento et al. (2014) Human umbilical cord tissue-derived MSCs (UCX®) attenuate remodeling following myocardial infarction by pro-angiogenic, anti-apoptotic and endogenous cell activation mechanisms”. Stem Cell Res. Ther. 10; 5(1):5.
Nourissat et al. (2010) Mesenchymal stem cell therapy regenerates the native bone-tendon junction after surgical repair in a degenerative rat model. PloS one. 2010:5 https://doi.org/10.1371/journal.pone.0012248.
Pei et al. (2013) Repair of large animal partial-thickness cartilage defects through intra-articular injection of matrix-rejuvenated synovium-derived stem cells. Tissue Eng. Part A. 19(9-10):1144-54.
Santos et al. (2015) 3D spheroid cell culture of umbilical cord tissue-derived MSCs (UCX®) leads to enhanced paracrine induction of wound healing”. Stem Cell Research & Therapy 6:90.
Santos et al. (2013) The role of human umbilical cord tissue-derived mesenchymal stromal cells (UCX®) in the treatment of inflammatory arthritis. J Transl Med., 17; 11(1):18.
Sekiya et al. (2015) Arthroscopic transplantation of synovial stem cells improves clinical outcomes in knees with cartilage defects. Clin. Orthop. Relat. Res. 473(7):2316-26.
WO2015/017772 A1, PCT/US2014/049395—STEM CELL-BASED PREPARATIONS AND METHODS FOR DERMAL AND CONNECTIVE TISSUE RECOSRUCTION AND REJUVENATION
WO 2009044379 A3, PCT/IB2008/054067—OPTIMIZED AND DEFINED METHOD FOR ISOLATION AND PRESERVTION OF PRECURSOR CELLS FROM HUMAN UMBILICAL CORD
WO/2014/203267—METHOD FOR ISOLATION, PURIFICATION AND INDUSTRIAL SCALE EXPANSION OF HUMAN ADIPOSE TISSUE DERIVED MESENCHYMAL STEM CELLS
WO/2014/203268, PCT/IN2013/000583—METHOD FOR ISOLATION, PURIFICATION AND INDUSTRIAL SCALE EXPANSION OF EQUINE ADIPOSE TISSUE DERIVED MENSENCHYMAL STEM CELLS
WO/2014/203269—METHOD FOR ISOLATION, PURIFICATION AND INDUSTRIAL SCALE EXPANSION OF CANINE ADIPOSE TISSUE DERIVED MESENCHYMAL STEM CELLS
EP 2110431—CARTILAGE REGENERATION USING HUMAN MESENCHYMAL STEM CELLS
EP 2626417—METHODS FOR CELL EXPANSION AND USES OF CELLS AND CONDITIONED MEDIA PRODUCED THEREBY FOR THERAPY.
EP 1276486; PCT/US2001/013267—JOINT REPAIR USING MESENCHYMAL STEM CELLS.
US 2004/0241144—CELL COMPOSITIONS FOR USE IN THE TREATMENT OF OSTEO-ARTHROSIS, AND METHODS FOR PRODUCING THE SAME.

1. A composition comprising mesenchymal stem/stromal cells (MSCs) derived from synovial tissues and umbilical cord tissues and wherein:
the MSCs are autologous and/or allogeneic mesenchymal stem/stromal cells;
the MSCs are obtained from adult, neonatal and/or foetal tissues;
the MSCs are integral and viable mesenchymal stem/stromal cells.

2. A composition according to claim 2 wherein the MSCs consist of MSCs derived from synovial tissues and umbilical cord tissues.

3. A composition according to any of the claims 1 or 2 wherein the MSCs are derived from the synovial membrane lining a mammal's metacarpophalangeal joint and/or from the Wharton's Jelly of the umbilical cord.

4. A composition according to any of the claims 1 to 3 wherein the MSCs are obtained from mammals, preferably from human, canine or equine cell-lines or tissues.

5. A composition according to any of the claims 1 to 4 further comprising paracrine factors obtained from growth media where MSCs have been cultured or are from blood derivatives, preferably from the umbilical cord blood plasma.

6. A composition according to claim 5 wherein the paracrine factors are selected from one or more of:
HGF in an amount of 10-250 pg/mL, preferably of 50-200 pg/mL, more preferably 75-150 pg/mL, even more preferably 100 pg/mL;
EGF in an amount of 10-250 pg/mL, preferably of 15-150 pg/mL, more preferably 25-100 pg/mL, even more preferably of 35-75 pg/mL, preferably of 50 pg/mL;
KGF in an amount of 10-250 pg/mL, preferably of 15-150 pg/mL, more preferably 25-100 pg/mL, even more preferably of 35-75 pg/mL, preferably of 50 pg/mL;
TGFβ-1 in an amount of 0.5-10.0 ng/mL, preferably of 1.0-7.5 ng/mL, more preferably of 2.5-5.0 ng/mL;
TGFβ-2 in an amount of 0.5-10.0 ng/mL, preferably of 0.75-7.5 ng/mL, more preferably of 1.0-2.5 ng/mL;
TGFβ-3 in an amount of 10-250 pg/mL, preferably of 50-200 pg/mL, more preferably of 100-150 pg/mL;
G-CSF in an amount of 10-250 pg/mL, preferably of 25-150 pg/mL, more preferably of 50-100 pg/mL;
VEGF-A in an amount of 10-250 pg/mL, preferably of 25-150 pg/mL, more preferably of 50-100 pg/mL;
FGF2 in an amount of 10-250 pg/mL, preferably 25-150 pg/mL, more preferably of 50-100 pg/mL;
LIF in an amount of 1.0-250 pg/mL, preferably of 10-150, more preferably 50-100 pg/mL;
IL-8 in an amount of 10-250 pg/mL, preferably 20-150 pg/mL, more preferably 40-100 pg/mL;
Eotaxin-1 in an amount of 10-250 pg/mL, preferably of 50-200 pg/mL, more preferably 100-125 pg/mL;
MCP-1 in an amount of 0.5-10.0 ng/mL, preferably of 0.75-7.5, more preferably of 1.0-5.0 ng/mL);
PDGF-BB in an amount of 0.5-10.0 ng/mL, preferably of 1.0-7.0 ng/mL, more preferably 2.5-5.0 ng/mL;
CCL5 in an amount of 0.5-10.0 ng/mL, preferably of 1.0-7.5, ng/mL more preferably of 3.0-5.0 ng/mL; and
sCD40L in an amount of 0.5-10.0 ng/mL, preferably of 1.0-7.5 ng/mL, more preferably of 2.0-40 ng/mL.

7. A composition comprising MSCs as described in any of the claims 1 to 6 for use as a medicament in humans or animals.

8. A composition according to claim 7 for use as a medicament to treat a musculoskeletal condition in humans or animals.

9. A composition according to claim 8 for use as a medicament to treat an osteoarthritis condition or any other condition etiologic to osteoarthritis in humans or animals.

10. A composition according to claim 8 for use as a medicament to treat a tendinopathy condition or any other condition etiologic to tendinopathy in humans or animals.

11. A method of treatment of a musculoskeletal condition, an osteoarthritis condition or any other condition etiologic to osteoarthritis, and/or a tendinopathy condition or any other condition etiologic to tendinopathy in humans or animals by using a composition as described in any of the claims 1 to 6.

12. A method of treatment according to claim 11 wherein the MSCs are autologous MSCs.

13. A process for obtaining a composition as described in any of the claims 1 to 6, comprising the following steps:
a) Providing a mammal body tissue producing mesenchymal stem/stromal cells;
b) Isolation of MSC cells;
c) Culturing the MSC cells of (b) by expansion and multiplication protocols;
d) Freezing and thawing the MSC cells of (c);
e) Formulating a composition comprising the MSC cells of (d).
